(19) United States
(12) Patent Application Publication
(10) Pub. No.: US 2016/0166368 A1
(43) Pub. Date: Jun. 16, 2016

(54) IMPLANTABLE PROSTHESIS

(71) Applicant: COUSIN BIOTECH, Wervicq Sud (FR)

(72) Inventors: Gilles SOLECKI, LANNOY (FR); Farid KAMCHE, TOURCOING (FR); Stéphane NOEL, HANTAY (FR)

(21) Appl. No.: 14/907,301

(22) PCT Filed: Jul. 24, 2014
(86) PCT No.: PCT/FR2014/051917

§ 371 Date: Jan. 25, 2016

(30) Foreign Application Priority Data
Jul. 25, 2013 (FR) 13 57362

(51) Int. Cl. A61F 2/00

(52) U.S. Cl. CPC A61F 2/0063; A61F 2/0077; A61F 2002/009; A61F 2002/0086; A61F 2002/0072

(57) ABSTRACT

The invention provides an implantable prosthesis (100) comprising:
an adhesive portion (102) that is capable of forming adhesions with organic tissues, the adhesive portion presenting a first peripheral edge (102a) and;
a non-adhesive portion (104) that inhibits the formation of adhesions with tissues, that is secured to the adhesive portion, and that presents a second peripheral edge (104a), the first peripheral edge (102a) being set back from the second peripheral edge (104a) so as to uncover the non-adhesive portion, in part, defining, between the first peripheral edge and the second peripheral edge, a non-adhesive peripheral band (106) that surrounds the adhesive portion (102);
the adhesive portion (102) has a central portion, and presents the shape of a star that includes a plurality of branches (108) that extend from the central portion, the adhesive portion having a plurality of notches (109) that are situated between the branches.

### BACKGROUND OF THE INVENTION

The present invention relates to the field of implantable medical devices.

More precisely, the invention relates to an implantable prosthesis that comprises:
an adhesive portion that is capable of forming adhesions with organic tissues, the adhesive portion presenting a first peripheral edge; and
a non-adhesive portion that inhibits the formation of adhesions with organic tissues, that is secured to the adhesive portion, and that presents a second peripheral edge, the first peripheral edge being set back from the second peripheral edge so as to uncover the non-adhesive portion, in part, defining, between the first peripheral edge and the second peripheral edge, a non-adhesive peripheral band that surrounds the adhesive portion.

Such a prosthesis is implanted, in particular, in the peritoneal cavity so as to repair the abdominal wall.

Traditionally, the adhesive portion is for placing against the peritoneum so as to be colonized by the human tissues, while the non-adhesive portion is configured to limit the adhesions that occur between the prosthesis and the abdominal organs after surgery.

WO 99/06079 describes such a composite prosthesis of rectangular shape in which the non-adhesive portion is constituted by a polysaccharide film of rectangular shape that projects laterally by several millimeters from the adhesive portion, which is made out of porous knitted fabric, so as to avoid contact between the viscera and the peripheral edge of the adhesive portion.

However, that prosthesis does not make it possible to avoid adhesions at the peripheral edge of the adhesive portion. Such adhesions appear since it is not possible to put the plane adhesive portion into contact uniformly with the concave surface of the peritoneum. Folds form in the prosthesis, thereby creating free adhesion zones in which the adhesive portion does not come into contact with the peritoneum. The viscera thus adhere to the free adhesion zones, and this complicates healing of the peritoneum.

GB 2 430 372 describes an implantable prosthesis having an adhesive portion that is constituted by a plurality of adhesive portions that are mutually disjoint and that extend radially around a sleeve.

US 2003/0212462 describes an implantable prosthesis having an adhesive portion in the shape of a heart.

### OBJECT AND SUMMARY OF THE INVENTION

An object of the invention is to propose an implantable prosthesis that remedies the above-mentioned drawbacks, and in which the risk of forming adhesions between the viscera and the adhesive portion is reduced still further.

The invention achieves its object by the fact that the adhesive portion has a central portion, and presents the shape of a star that includes a plurality of branches that extend from the central portion of the adhesive portion, the adhesive portion including a plurality of notches, each notch being situated between two adjacent branches.

As a result of its star shape, the adhesive portion is better at fitting closely to the concave surface of the peritoneum, thereby substantially reducing, or indeed avoiding, the formation of free adhesion zones.

In other words, the branches deform so as to match the concave shape of the peritoneum. Since the branches are in contact with the peritoneum, the adhesive portion does indeed remain in contact with the peritoneum, and in particular along the peripheral edge of the adhesive portion. In this respect, it should be understood that the adhesive and non-adhesive portions are flexible.

It should also be understood that the non-adhesive portion also projects laterally from the periphery of the adhesive portion, whenever the first peripheral edge of the adhesive portion is set back from the second peripheral edge.

The branches of the adhesive portion touch one another as a result of them extending from the central portion of the adhesive portion.

Preferably, the minimum width of the non-adhesive peripheral band lies in the range 3 millimeters (mm) to 6 mm.

Preferably, the number of branches lies in the range four to ten, and is preferably six.

Advantageously, each branch extends radially over a height, and it tapers from the central portion of the adhesive portion towards its vertex. Preferably, the vertex is rounded. Also preferably, the base of the branch is wider than its vertex.

The branches preferably present the same height. However, without going beyond the ambit of the present invention, some ribs could present heights that are greater than others.

Preferably, the branches present a height that lies in the range 3 mm to 50 mm. Such a range of heights makes it possible to shape the branches and the adhesive portion closely to the peritoneum. Preferably, the width of the non-adhesive peripheral band lies in the range 3 mm to 10 mm at the vertices of the branches.

Advantageously, the central portion presents a span that preferably lies in the range 40 mm to 90 mm.

In an advantageous embodiment, the branches are formed integrally with the central portion. To do this, and by way of example, the adhesive portion is obtained by cutting a porous knitted fabric into the shape of a star.

In a variant, the branches are separate pieces that are fastened to the central portion.

Advantageously, the central portion presents a shape that is circular or oval. The radius of the central portion is preferably greater than or equal to the height of the branches of the adhesive portion.

Preferably, the adhesive portion is made out of a textile material. By way of example, but not exclusively, the textile material is a warp knitted fabric of Chain or Raschel type. The adhesive portion is preferably constituted, at least in part, by Poly-L-lactic add (PLLA).

In a variant, the branches are made out of a textile material that is more rigid than the textile material that constitutes the central portion.

In an embodiment, the non-adhesive portion presents the shape of a disk or of an oval.

The disk or oval shape makes it possible to match the concave shape of the peritoneum better than the rectangular shape of the prior art.

In another particularly advantageous embodiment, the non-adhesive portion also presents the shape of a star, and the branches of the non-adhesive portion are superposed with branches of the adhesive portion.

Thus, the adhesive portion includes at least as many branches as the non-adhesive portion.

An advantage of the star shape of the non-adhesive portion is to improve still further the shaping of the prosthesis to the concave shape of the peritoneum. The non-adhesive portion is also prevented from forming folds between its branches, in which organic tissues could accumulate.

Once again, the branches of the non-adhesive portion project laterally from the branches of the adhesive portion, so as to define the non-adhesive peripheral band. Preferably, the minimum width of the non-adhesive peripheral band lies in the range 5 mm to 15 mm between two adjacent branches.

In this advantageous embodiment, it should thus be understood that the implantable prosthesis presents the shape of a star.

Advantageously, the non-adhesive portion includes a central portion from where the branches of the non-adhesive portion taper.

Still preferably, the vertices of the branches of the non-adhesive portion are rounded.

In an advantageous variant, the adhesive portion is made out of knitted fabric, while the non-adhesive portion is a polymer film. Preferably, but not exclusively, the polymer film is a resorbable polymer of lactic acid.

The film is fastened to the knitted fabric by stitching or by melting the monofilaments onto the yarn.

In order to improve the positioning of the prosthesis in the body during surgery, it further includes removable expansion means. By way of example, the expansion means may be an inflatable balloon that the surgeon may inflate by means of a tube. The inflatable balloon is preferably housed in a pouch that is fastened to the adhesive portion. The pouch preferably presents the shape of a thick disk, and it includes a wall made out of knitted fabric that is fastened to the adhesive portion and that is provided with an orifice that makes it possible to remove the balloon after the prosthesis has been implanted.

The prosthesis is plane, or, in an advantageous variant, it presents a convex shape for fitting more closely to the concave shape of the peritoneum.

### BRIEF DESCRIPTION OF THE DRAWINGS

The invention can be better understood on reading the following description of embodiments of the invention, given by way of non-limiting example, and with reference to the accompanying drawings, in which:

FIG. 1 is a plan view of a first embodiment of an implantable prosthesis of the invention, in which the adhesive portion presents a star having six branches, while the non-adhesive portion presents the shape of a disk;

FIG. 2 is a side view of the FIG. 1 prosthesis;

FIG. 3 shows the FIG. 1 prosthesis positioned in contact with the peritoneum;

FIG. 4 is a plan view of a second embodiment of an implantable prosthesis of the invention, in which the non-adhesive portion also presents the shape of a star having six branches;

FIG. 5 is a side view of the FIG. 4 prosthesis;

FIG. 6 shows a variant of the FIG. 4 prosthesis, in which the branches of the adhesive portion are separate;

FIG. 7 shows a variant of the FIG. 4 prosthesis, in which each of the adhesive and non-adhesive portions include eight branches; and

FIGS. 8 and 9 show another embodiment of the FIG. 4 prosthesis, which further includes an expansion balloon.

### DETAILED DESCRIPTION OF THE INVENTION

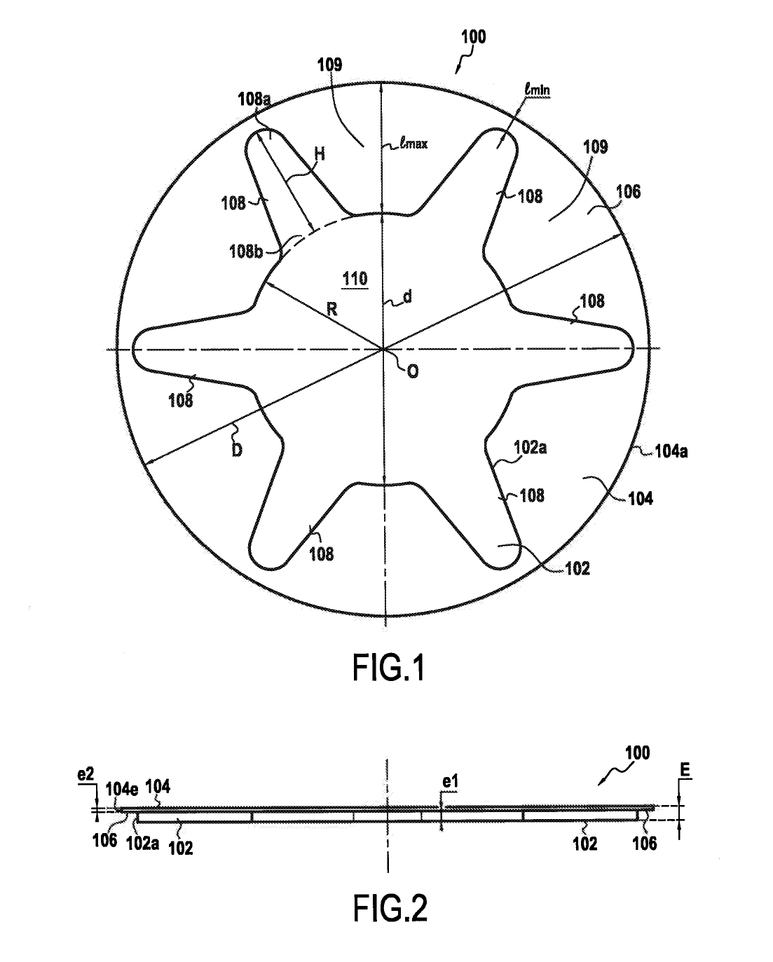

A first embodiment of an implantable prosthesis 100 of the present invention is described with reference to FIGS. 1 to 3.

In this embodiment, the implantable prosthesis 100 is an intraperitoneal prosthesis. This type of prosthesis is also known as an intraperitoneal visceral plate. The prosthesis is flexible so as to shape itself to the concave shape of the peritoneum.

FIG. 1 is a plan view of the implantable prosthesis 100. The implantable prosthesis presents the general shape of a disk of center O, having a diameter D that is about 100 mm. The prosthesis 100 presents a thickness E of about 500 microns (μm).

The prosthesis 100 is of the composite type and comprises an adhesive portion 102 secured to a non-adhesive portion 104.

In traditional manner, the adhesive portion 102 is designed to form adhesions with organic tissues, while the non-adhesive portion 104 is designed to inhibit the formation of adhesions with organic tissues.

After being implanted in the human body, the adhesive portion 102 comes into contact with the peritoneum so as to adhere thereto, while the non-adhesive portion 104 forms a barrier that comes into contact with the viscera so as to prevent the viscera from adhering with the peritoneum during the healing period.

In this embodiment, the adhesive portion is a warp knitted fabric of Chain or Raschel type. Preferably, the knitted fabric comprises a resorbable portion and a non-resorbable portion. In a variant, the knitted fabric could be completely non-resorbable.

In this embodiment, the knitted fabric comprises a resorbable portion made with a PLLA monofilament of about 200 decitex (dtex), and a non-resorbable portion made with a polypropylene monofilament of about 70 dtex.

In this first embodiment, the non-adhesive portion 104 is a resorbable polymer film of lactic acid. The polymer film is fastened to the knitted fabric by stitching or by melting the monofilaments onto the film.

In this embodiment, the adhesive portion 102 presents a thickness e1 of about 475 μm, while the non-adhesive portion 104 presents a thickness e2 of about 25 μm.

With reference once again to FIG. 1, it should be observed that the adhesive portion 102 presents a first peripheral edge 102a. The non-adhesive portion 104, disk shaped in this embodiment, includes a second peripheral edge 104a that is circular.

As can be seen in FIGS. 1 and 2, the first peripheral edge 102a of the adhesive portion 102 is set back from the second peripheral edge 104a of the non-adhesive portion 104 so as to uncover the adhesive portion, in part, defining, between the first peripheral edge 102a and the second peripheral edge 104a, a non-adhesive peripheral band 106 that surrounds the adhesive portion 102.

In FIG. 2, it should be observed that the non-adhesive peripheral band 106 is in the same plane as the non-adhesive portion 104, and presents the same thickness e2.

It should also be observed that the non-adhesive peripheral band 106 does not cover the periphery of the adhesive portion 102.

The non-adhesive peripheral band 106, defined between the first peripheral edge 102a and the second peripheral edge 104a, presents a width l that is defined between the first peripheral edge 102a and the second peripheral edge 104a.

In FIG. 1, the minimum radial width of the non-adhesive peripheral band 106 is referred to as lmin, and the maximum width of the non-adhesive peripheral band 106 is referred to as lmax.

In accordance with the invention, the adhesive portion 102 presents the shape of a star that includes a plurality of branches 108. In this embodiment, the adhesive portion includes six branches 108. Each of the branches 108 extends radially from a central portion 110 of the adhesive portion.

The adhesive portion further includes a plurality of notches 109, each of the notches 109 being situated between two adjacent branches.

The branches touch one another as a result of them extending from the central portion 110 of the adhesive portion. The adhesive portion thus extends continuously between the various branches. Each branch 108 extends radially over a height H, and presents a vertex 108a and a base 108b. Each branch tapers from the central portion towards its vertex. In this embodiment, the branches present the same height H of about 20 mm. The base of each branch is wider than its vertex. Each branch thus includes a portion that converges towards its vertex. Furthermore, it should be observed that the vertices of the branches are rounded.

The central portion 110 from which the branches extend presents the general shape of a disk having a diameter d that is about 60 mm in this embodiment.

It should be observed in FIG. 1 that the center of the disk-shaped non-adhesive portion substantially coincides with the center of the circular-shaped central portion. In addition, the prosthesis presents central symmetry of center O.

In FIG. 1, it should also be observed that the minimum width lmin of the non-adhesive peripheral band 106 is situated at the vertices 108a of the branches 108. In this embodiment, the width lmin, corresponding to the minimum set back, is about 5 mm.

It should also be observed that the maximum width lmax, corresponding to the maximum set back, is situated between two consecutive branches, and is about 40 mm.

In the embodiment in FIG. 1, the branches 108 are formed integrally with the central portion 110.

It should also be observed that the radius R of the central portion is slightly greater than the height H of the branches of the adhesive portion. Also, the branches are small compared to the central portion.

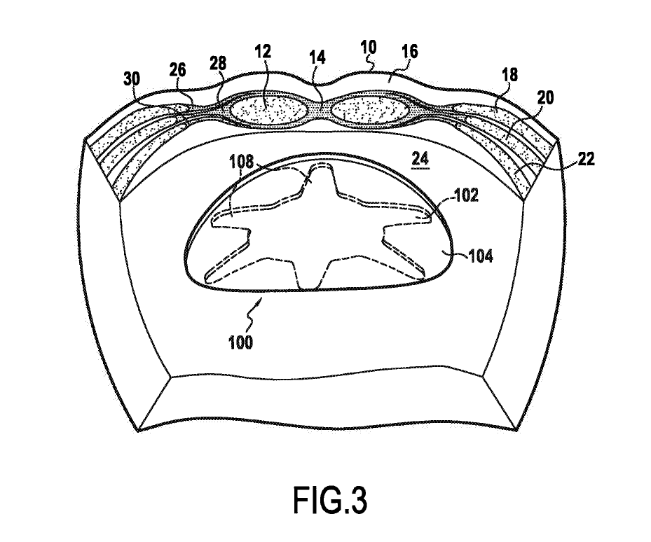

The positioning of the prosthesis 100 in the peritoneal region of the patient is explained below with reference to FIG. 3.

This figure is a diagram showing a portion of the abdomen, in a section taken above the navel.

In FIG. 3, the following references are used:
10: skin;
12: rectus muscle of the abdomen;
14: white line;
16: sub-cutaneous tissue (fatty layer);
18: external oblique muscle;
20: internal oblique muscle;
22: transverse muscle of the abdomen;
24: peritoneum;
26: aponeurosis of the external oblique muscle;
28: aponeurosis of the internal oblique muscle;
30: aponeurosis of the transverse muscle of the abdomen.

As can be seen in FIG. 3, the prosthesis adopts the concave shape of the peritoneum 24, and it remains uniformly in contact with the peritoneum after its placement on the surgical site.

In conventional manner, for being put into place, the prosthesis takes the shape of an umbrella while it is being transferred inside the peritoneum by the surgeon, and it is then deployed so as to come into contact with the peritoneum.

The prosthesis of the FIG. 3 embodiment seeks, in particular, to repair hernias of the white line facing the two rectus muscles of the abdominal wall, or even umbilical hernias.

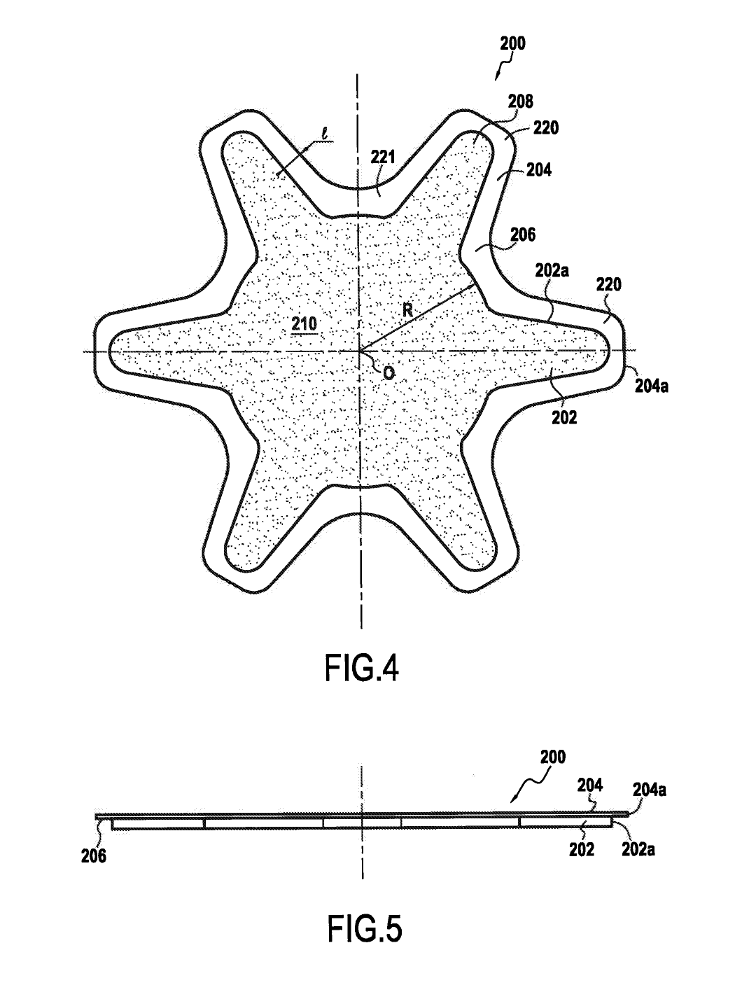

A second embodiment of the prosthesis 200 of the invention is described below with reference to FIGS. 4 and 5.

The second embodiment of the implantable prosthesis of the present invention differs from the first by the fact that the non-adhesive portion 204 also presents the shape of a star.

In the embodiment in FIG. 4, the non-adhesive portion 204 presents six branches 220 that are superposed with the branches 208 of the adhesive portion. The non-adhesive portion includes a central portion 221 from where the branches 220 of the non-adhesive portion taper radially. The branches of the non-adhesive portion taper towards their respective vertices, the vertices being rounded.

It should also be observed that the non-adhesive peripheral portion 206 that surrounds the adhesive portion 202 presents a width l, of about 5 mm, that is substantially constant over the entire periphery of the prosthesis 200.

It should be remembered that the non-adhesive peripheral portion 206 is defined between the first peripheral edge 202a of the adhesive portion 202, and the second peripheral edge 204a of the non-adhesive portion 204.

In the embodiment in FIG. 4, the width e of the non-adhesive peripheral band lies in the range 3 mm to 10 mm, and this over the entire periphery of the prosthesis.

In the second embodiment, the branches 208 of the adhesive portion 202 are formed integrally with the central portion 210.

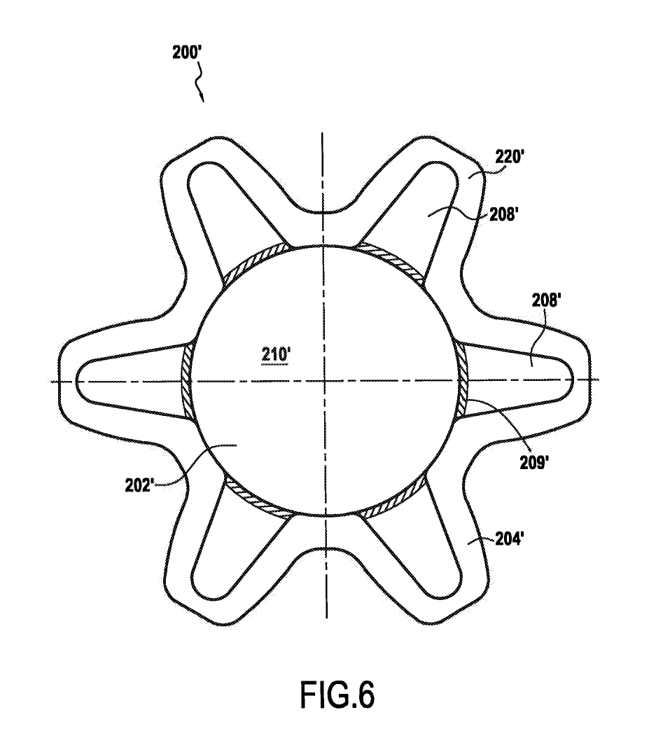

In the variant in FIG. 6, the branches 208′ of the adhesive portion 202 are separate pieces that are fastened to the central portion 210′. The branches 208′ of the adhesive portion 202 are made out of a textile material that is more rigid than the textile material that constitutes the central portion 210′. In the embodiment in FIG. 6, the branches 208′ are heat-sealed, by means of heat-seals 209′ to the central portion 210′. However, it is possible to envisage some other fastener means without going beyond the ambit of the present invention.

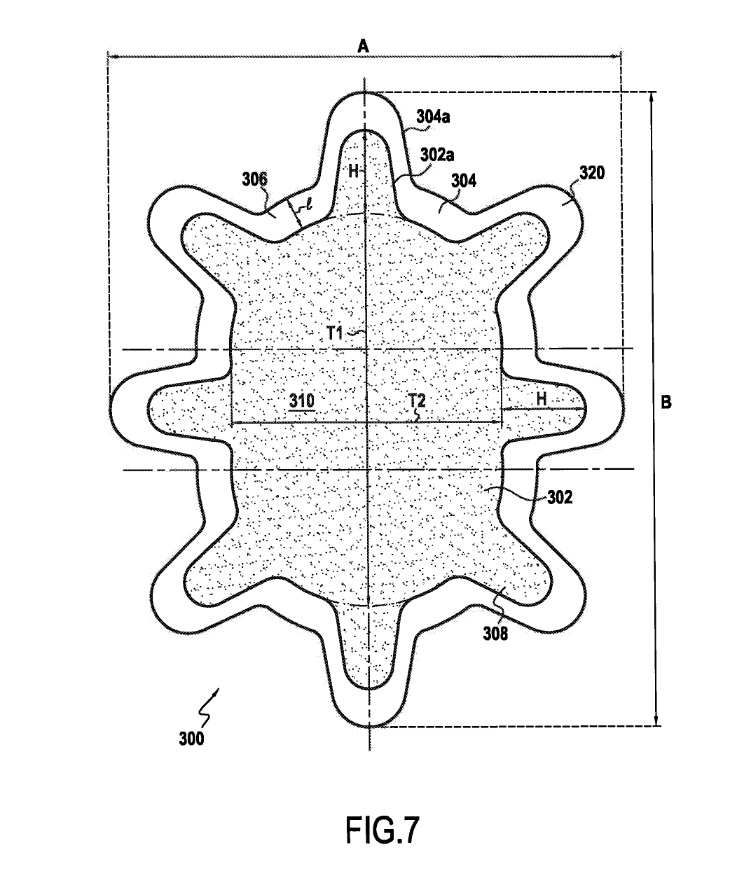

The implantable prosthesis 300 shown in FIG. 7 is a variant of the FIG. 4 implantable prosthesis.

In this variant, the central portion 310 of the adhesive portion 302 presents a shape that is substantially oval. The longitudinal and transverse dimensions are referenced T1 and T2 in FIG. 7. It should be observed that the half-width of the central portion, considered along the transverse dimension T2, is greater than the height H of the branches. Also, the branches are likewise small compared to the central portion.

In addition, the adhesive portion 302 includes eight branches 308. The non-adhesive portion 304 also includes eight branches 320. The width A of the prosthesis 300 is about 125 mm, while the length B is about 155 mm.

Naturally, other star shapes of the adhesive and non-adhesive portions could be envisaged, together with a different number of branches.

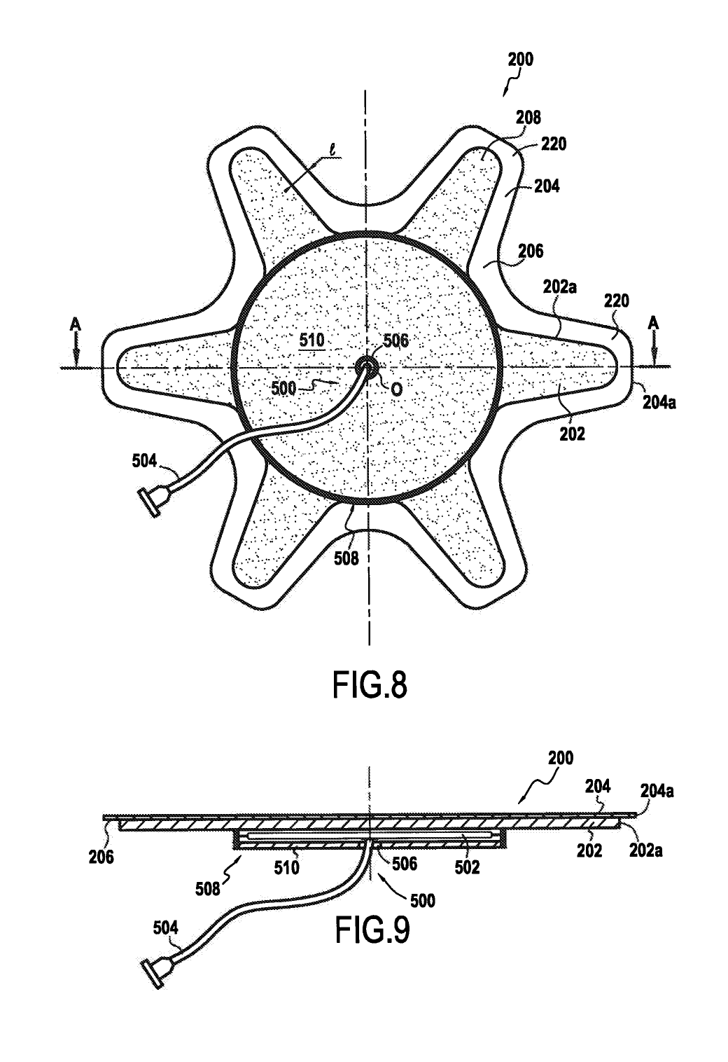

In the embodiment in FIGS. 8 and 9, the prosthesis 200 further includes removable expansion means 500 that are in the form of an inflatable balloon 502.

The balloon 502 is connected to the end of an inflatable tube 504. In the deflated state, the balloon presents the shape of a flexible pellet that is for inserting, via an orifice 506, inside a pouch 508 that is fastened to the adhesive portion 202 of the prosthesis 200. The pouch 508 includes an outer wall 510 that is fastened to the adhesive portion 202. In this embodiment, the outer wall 510 is made out of knitted fabric, and it is stitched to the knitted fabric of the adhesive portion. However, fastening by heat sealing could be provided. As can be seen in FIG. 8, in this embodiment, the outer wall 510 presents the shape of a disk that is inscribed inside the star-shaped adhesive portion 202.

When fluid is pumped through the tube 504, the balloon inflates, thereby giving the prosthesis 200 a convex shape, and improving its shaping to the concave shape of the peritoneum. After implanting the prosthesis, the balloon is removed via the orifice 506, after being deflated beforehand.

1-15. (canceled)

16. An implantable prosthesis comprising:
an adhesive portion configured to adhere to organic tissues, the adhesive portion presenting a first peripheral edge; and
a non-adhesive portion that inhibits adhesions with organic tissues, that is secured to the adhesive portion, and that presents a second peripheral edge, the first peripheral edge being set back from the second peripheral edge so as to uncover the non-adhesive portion, in part, defining, between the first peripheral edge and the second peripheral edge, a non-adhesive peripheral band that surrounds the adhesive portion;
the adhesive portion having a central portion, and presenting the shape of a star that includes a plurality of branches that extend from the central portion of the adhesive portion, the adhesive portion including a plurality of notches, each notch being situated between two adjacent branches.

17. The prosthesis according to claim 16, wherein each branch extends radially over a height, and tapering from the central portion of the adhesive portion towards a vertex.

18. The prosthesis according to claim 17, wherein the branches present a height that lies in a range of 3 mm to 50 mm.

19. The prosthesis according to claim 17, wherein the width of the non-adhesive peripheral band lies in a range of 3 mm to 10 mm at the vertices of the branches.

20. The prosthesis according to claim 16, wherein the branches are formed integrally with the central portion.

21. The prosthesis according to claim 16, wherein the branches are separate pieces that are fastened to the central portion.

22. The prosthesis according to claim 16, wherein the central portion presents a shape that is circular or oval.

23. The prosthesis according to claim 16, wherein the number of branches lies in a range of four to ten.

24. The prosthesis according to claim 16, wherein the adhesive portion comprises a textile material.

25. The prosthesis according to claim 21, wherein the adhesive portion comprises a textile material and wherein the branches comprise a second textile material that is more rigid than the textile material of which the central portion is comprised.

26. The prosthesis according to claim 16, wherein the non-adhesive portion presents the shape of a disk or of an oval.

27. The prosthesis according to claim 16, wherein the non-adhesive portion presents the shape of a star, and wherein the branches of the non-adhesive portion are superposed with branches of the adhesive portion.

28. The prosthesis according to claim 27, wherein the non-adhesive portion includes a central portion from which the branches of the non-adhesive portion taper.

29. The prosthesis according to claim 16, wherein the adhesive portion comprises a knitted fabric, while the non-adhesive portion comprises a polymer film.

30. The prosthesis according to claim 16, further including a removable expansion device.
